(19) United States
(12) Patent Application Publication
(10) Pub. No.: US 2020/0390563 A1
(43) Pub. Date: Dec. 17, 2020

(54) MEDICAL ARTHRODESIS IMPLANT

(71) Applicant: ADSM, Wettolsheim (FR)

(72) Inventors: Christophe AVEROUS, Mittelhausbergen (FR); Patrice DETERME, Toulouse (FR); Christophe CERMOLACCE, Marseille (FR); Patrice DIEBOLD, Nancy (FR); Stéphane GUILLO, Merignac (FR); Thierry HECKEL, Lyon (FR); Hubert ROCHER, Pessac (FR); Renaud FRIOUX, Vannes (FR); Christophe ROY, Bourg-de-Peage (FR); Danilo CAMPANI, Wettolsheim (FR)

(21) Appl. No.: 16/754,445

(22) PCT Filed: Oct. 5, 2018
(86) PCT No.: PCT/FR2018/052462

§ 371 Date: Apr. 27, 2020

Related U.S. Application Data
(60) Provisional application No. 62/570,156, filed on Oct. 10, 2017.

(30) Foreign Application Priority Data
Oct. 9, 2017 (FR) 1759426

(51) Int. Cl. A61F 2/42

(52) U.S. Cl. CPC A61F 2/4225; A61F 2002/30622; A61F 2/4241

(57) ABSTRACT

The medical implant (1) according to the present invention comprises a first, generally elongate portion (20) extending along the longitudinal axis A, intended to be inserted into the first bone (P1) and comprising first attachment means (24), and a second, also elongate, portion (30) following another longitudinal axis B, intended to be inserted into the second bone (P2) and comprising second attachment means (34), wherein said first and second portions (20, 30) are connected together via a connecting bridge (40) which is centered about the orthonormal axes X, Y, Z of said implant (1), providing for at least one axial offset of the second portion (30) with respect to the first portion (20) such that the longitudinal axis B of the second portion (30) is positioned in at least one direction forming an angle (α) relative to the longitudinal axis A of the first portion (20) and is offset by at least a certain distance along at least one of the axes XX′, YY′ and ZZ′ of the connecting bridge (40) in order to avoid any offset between the outer upper portions of the first bone (P1) and the second bone (P2).

This invention relates to a medical implant intended to act as a connection between two bones, one adjacent to a joint. According to one non-exhaustive example, the invention is based on a medical implant that connects and attaches two adjacent proximal or distal foot or hand phalanges to one another.

Certain pathologies, like claw toes, hallux valgus or metatarsalgia, cause toe deformation: in particular, sometimes two adjacent phalanges of one toe lose their natural alignment and form an angle that deforms the toe. This kind of deformation can be vertical, which causes metatarsalgia in the patient: the toe takes on a “claw” form in which it permanently folds over itself. The deformation can also be horizontal, which is hallux valgus: the toe, particularly the big toe, protrudes outward.

These pathologies cause pain in the front foot, calluses, and functional discomforts. The patient will have trouble walking correctly, and may also have difficulties wearing shoes.

To correct them, common practice is to use an inter-phalangeal arthrodesis: this technique surgically blocks the articulation formed by both phalanges that have lost their natural alignment. It realigns them by fusing both bones together, making both phalanges stuck to one another for as long as needed for the bones to fuse using an implant.

The implant used in this type of correction comprises two elements: one intended to be inserted in a first phalange, for example a proximal phalange, and a second part intended to be inserted in a second phalange, for example the distal phalange adjacent to the first phalange: both phalanges then become attached to one another. The implant must allow both attached bones, or phalanges, to fuse.

Implants intended to perform interphalangeal arthrodesis have already been described.

Document WO2011/110784 describes an implant comprising a first part intended to be inserted in the proximal phalange and a second part intended to be inserted in the distal phalange.

According to European patent EP 2544633, belonging to the applicant, there is also a medical implant comprising a first part that is elongate, supported by a longitudinal axis, intended to be inserted in the first bone and comprising attachment means, and a second part, also elongate, which has another longitudinal axis, intended to be inserted in the second bone and comprising two attachment means. The first and second parts are connected by a central, star-shaped hub with at least three branches.

In any case, existing implants do not provide the correct alignment of the outer parts of the phalange. This creates, for example, a step between the proximal and distal phalanges. This can cause particular damage if there is arthrodesis of the foot's bones, to the extent where this kind of step can conflict with the shoe. The patient can feel pain or difficulties putting on shoes.

To avoid these inconveniences, after the insertion, surgeons generally cut the bone or the part of the phalange that create the step. Generally, the surgeon cuts the upper part of the distal phalange bone. This resection is traumatic for the patient.

Thus, the implant according to the present invention allows the fusion of a proximal phalange and a distal phalange, for the foot or the hand, without creating this step or misalignment after insertion.

The present invention is an implant with a specific structure, allowing the fusion of a proximal phalange and a distal phalange for the hand or the foot, preventing the outer upper parts between both phalanges from becoming misaligned. That way, after the implant is inserted according to the invention, both phalanges are perfectly aligned, and the surgeon will not need to resect the upper part of the bone of the distal phalange.

The implant according to the invention therefore makes the operation to fuse both phalanges simpler. Additionally, the implant according to the invention makes the operation much less traumatizing for the patient. The implant according to the invention also greatly reduces the risk of complications after the operation. The implant according to the invention also makes it possible to restore the organ treated to its natural anatomy, particularly the natural angle between the phalanges that have been fused.

The medical implant according to this invention is meant to allow bone fusion between a first bone (P1) and a second bone (P2), the said implant includes a first part that is elongate supported by a longitudinal axis A, intended to be inserted in the first bone (P1), including the first attachment means, and a second part, also elongate, which has another longitudinal axis B, intended to be introduced in the second bone (P2), including second attachment means. The said first and second parts are connected to one another via a connecting bridge, the said connecting bridge is centered around the orthonormal axes X, Y, Z of the said implant, providing at least an axial offset of the second part with regard to the first part. This is so that the longitudinal axis B of the said second part is partially positioned in at least one direction, which is inclined at an angle (α) with regard to the longitudinal axis A of the first part, and another part offset by at least the distance of the length of at least one of the axes XX′, YY′ and ZZ′ of the connecting bridge, in order to prevent the outer upper parts between the first bone (P1) and the second bone (P2) to become misaligned.

Indeed, the profile of the connecting bridge can allow different axial offsets, with an option to combine, in order to place the second part in different geometric planes, which can also be combined.

The combination of the angular and linear offset with regard to the center of the joint best solves the technical problem since the surgeon no longer has to use the surgical saw blade to even out the heights of P1 and P2 once the implant is in place.

The medical implant according to the invention includes a longitudinal axis B of the second part which is offset by a distance (Dₛ₁) along axis YY′.

The medical implant according to the invention includes a longitudinal axis B of the second part, which is offset by a distance (Dₛ₂) along axis ZZ′.

The medical implant according to this invention includes a first part which is elongate and comprises at least three brackets, each extending respectively along longitudinal axis A and including a distal end and a proximal end.

The medical implant following this invention comprises a first part, in which the brackets are regularly arranged around the longitudinal axis A, making it possible to drill a through-hole or a cannula between the said brackets and in the body of the first part supported by the longitudinal axis A and crossing the connecting bridge, which would allow a guide pin to go through.

The medical implant according to this invention comprises a second part, in which each bracket includes attachment means on its edge made of hooked notches creating a toothed outside profile all along the first part, the said hooked notches each pointing toward the connecting bridge.

The medical implant according to this invention comprises a second part, which is also extended and constitutes at least three brackets that each respectively extend according to the longitudinal axis B and includes a distal end and a proximal end.

The medical implant according to this invention includes a longitudinal axis A of the first part that cuts the longitudinal axis B of the second part at an intersection point I₃ located between the distal and proximal ends of the said first part.

The medical implant according to this invention comprises a point of intersection I₃, located at a distance L₃ from the proximal end and a distance L₃ from the distal end of the first part.

The medical implant according to this invention includes the orthonormal axes X, Y, Z of the connecting bridge, which intersect at an intersection point I₂.

The medical implant according to this invention comprises a point of intersection I₂ which is located at a distance L₂ from the proximal side of the second part.

The medical implant according to this invention comprises a connecting bridge for which the vertical axis D cuts the longitudinal axis B of the second part at an intersection point I₁ found at a distance Dₛ₁ from the point of intersection I₂.

The medical implant according to this invention comprises a connecting bridge for which the longitudinal axis XX′ coincides with the longitudinal axis A of the first part.

The medical implant according to this invention comprises a connecting bridge for which the longitudinal axis XX′ is tilted with regard to the longitudinal axis A of the first part.

The medical implant according to this invention comprises a connecting bridge bordered by two opposite faces that coincide respectively with the distal end of the first part and the proximal end of the second part.

The medical implant according to this invention comprises a distance (Dₛ₁) which is less than or equal to 20 millimeters.

The medical implant according to this invention comprises a distance (Dₛ₂) which is less than or equal to 10 millimeters.

The description below regarding the drawings attached, given only as examples, will help better understand the invention, its characteristics and the advantages it has:

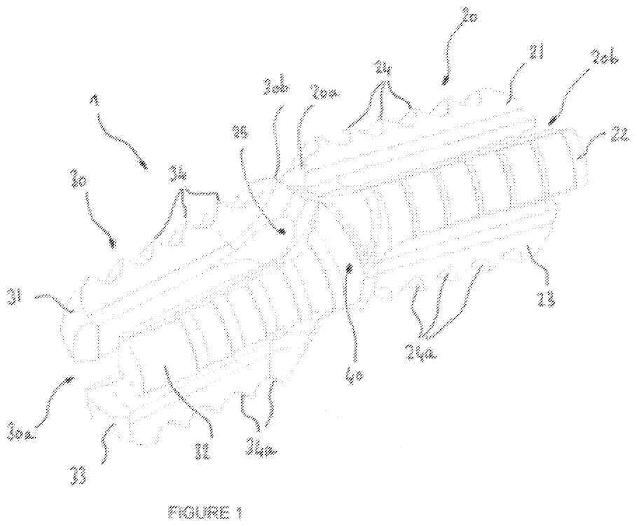

FIG. 1 is a perspective view that illustrates the medical implant according to this invention.

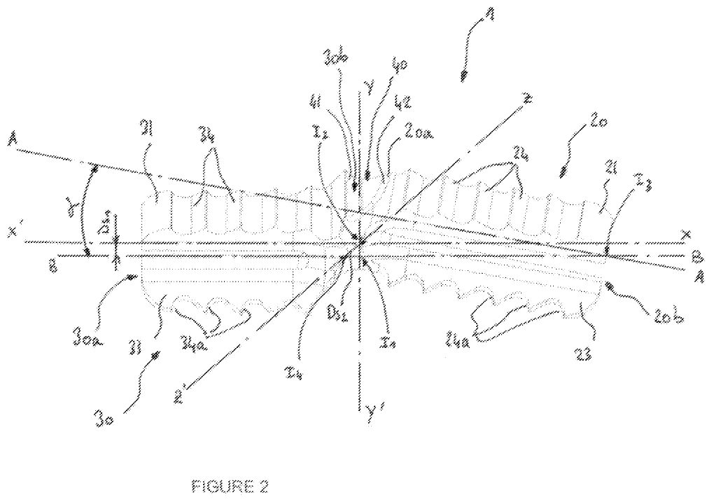

FIG. 2 is a front view showing the medical implant according to this invention.

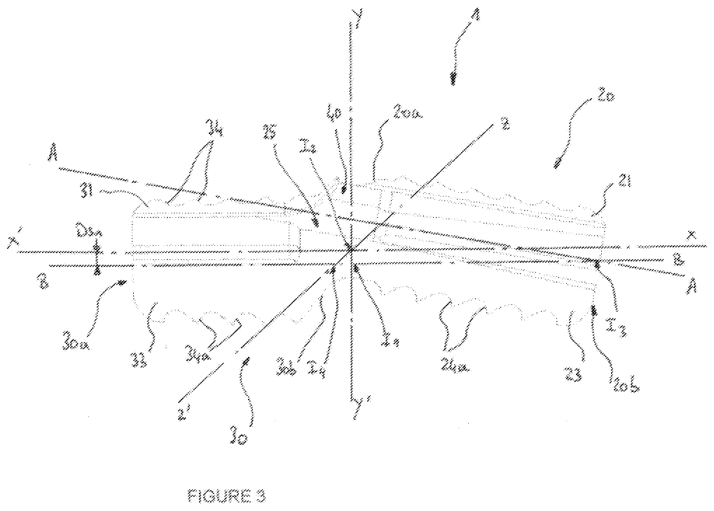

FIG. 3 is a cut view showing the medical implant according to this invention.

We showed in FIGS. 1 to 3 a medical implant 1 according to the invention which would allow the creation of an arthrodesis in order to connect and fuse two adjacent phalanges between them, called proximal P1 and distal P2.

The medical implant according to the invention 1 is preferably a one-piece unit. It can be made, for example, by injecting molding or cutting. The medical implant 1 can be made of a biocompatible material, metallic or not. For example, this material can be chosen from polyaryletherketones (PEEK or PEKK), titanium, stainless steel, polylatic acids and their mixtures.

The implant 1 comprises a first part 20 and a second part 30 meant to be respectively inserted in the medullary canal of the proximal phalange P1 and the distal phalange P2, the said first and second parts 20, 30 being connected to one another by the connecting bridge 40 centered around the orthonormal axes X, Y, Z of the said implant 1.

The first part 20 is extended, including a distal end 20a and a proximal end 20b. The first part 20 has a longitudinal axis A intended to be aligned, or coinciding with, when inserted into the longitudinal axis of the medullary canal of the proximal phalange P1.

The first part 20 comprises at least three brackets 21, 22, 23 each extending respectively along the longitudinal axis A and from the distal end 20a or the connecting bridge 40. Brackets 21, 22, 23 give the first part 20 an overall star-shaped cylinder shape.

In the example shown, the three brackets 21, 22, 23 extend in parallel one over the other. In an embodiment not shown, the brackets 21, 22, 23 can extend based on the respective axes that are slightly separate from the longitudinal axis A.

The brackets 21, 22, 23 are regularly placed around the longitudinal axis A, making it possible to drill a bore or canula 25 between the said brackets and in the body of the first part 20, supported by the longitudinal axis A and crossing the connecting bridge 40 for a guide pin to fit and be able to better handle and guide the implant 1 during its insertion.

The bore hole or canula 25 also makes it possible to insert an osteo-integration activator in the implant 1.

According to the invention, each bracket 21, 22, 23 has hooked notches 24 on its outside edges which creates a toothed outer profile on the whole length of the first part 20.

The hooked notches 24 are the first attachment means intended to hold the first part 20 of the implant 1 in the proximal phalange P1 in which the implant is meant to be inserted.

The hooked notches 24 each have a ridge 24a pointing toward the connecting bridge 40, which makes it possible to improve the fixation and holding of the first part 20 inside the medullary canal of the proximal phalange P1.

The second part 30 is elongate, comprising a distal end 30a and a proximal end 30b. The second part 30 is intended to be inserted in the medullary canal of the distal phalange P2 adjacent to the said proximal phalange P1.

The second part has a longitudinal axis B meant to be aligned, or coinciding with, the longitudinal axis of the medullary canal of the distal phalange P2.

The second part 30 comprises at least three brackets 31, 32, 33 each extending respectively along the longitudinal axis B and from the distal end 30a or the connecting bridge 40.

The brackets 31, 32, 33 each give the second part 30 a slightly conic star shape. In the example shown, the three brackets 31, 32, 33 extend in parallel over one another and in the alignment of the brackets 21, 22, 23 of the first part 20. In an embodiment not shown, brackets 31, 32, 33 can extend along the respective axis, slightly separating from the longitudinal axis B.

According to the invention, each bracket 31, 32, 33 includes on its outer edges the hooked notches that create a toothed external profile all along the second part 30.

The hooked notches 34 are the second attachment means intended to hold the second part 30 of the implant 1 in the distal phalange P2 in which the implant is meant to be inserted.

The hooked notches 34 each have a ridge 34a pointing toward the connecting bridge 40 which helps better fix and hold the second part 30 inside the medullary canal of the distal phalange P2.

The direction of the hooked notches 24 and 34 of each first and second part 20, 30 respectively pointing toward to the connecting bridge 40 makes it possible when inserting the implant 1 to more easily bring together the proximal P1 and distal P2 phalanges to fuse them together.

The connecting bridge 40 is centered around the orthonormal axes X, Y, Z, which allows the bridge to have an axial offset of the second part 30 with regard to the first part 20 of the implant 1. The axes do not intersect centrally. Because of this and to adapt to the anatomical needs, the implant is bayonet-shaped.

The connecting bridge 40 may allow different axial offsets, combined or not, in order to position the second part 30 with regard to the first part 20 in different geometrical planes, which can be combined. The axes are offset with regard to the center of the joint.

This embodiment of the implant 1 makes it possible to offset the distal phalange with regard to the proximal phalange toward the inside or outside of the foot or hand and thus to respectively adduct or abduct it. This embodiment is particularly advantageous for treating a Hallux Valgus.

The connecting bridge 40 is surrounded by two opposite faces 40, 41, which, depending on its geometric profile, could respectively coincide with the distal end 20a of the first part 20 and the proximal end 30b of the second part 30.

In the embodiment example of this invention, the profile of the connecting bridge 40 makes it possible to position the longitudinal axis B of the second part 30 in a first direction which is titled at angle α with regard to the longitudinal axis A of the first part 20.

Angle α may vary by about 0 to 25°, for example from 10 to 20°, preferably 10°. It is possible to vary the angle α, in particular to adjust the implant from the invention to the anatomy of the human body part to treat, for example, the foot or the hand.

Also, the profile of the connecting bridge 40 makes it possible to position the longitudinal axis B of the said second part 30 in a second direction, which is offset by a distance Dₛ₁, with regard to the longitudinal axis XX′ of the said connecting bridge 40.

In our example, the distance Dₛ₁ runs along the axis YY′. Depending on the profile of the connecting bridge 40, it is also possible to position the longitudinal axis B of the second part 30 in a third or fourth direction, which is offset by a distance Dₛ₂ along the longitudinal axis ZZ′ of the said connecting bridge 40.

Depending on the pathological case, the implant 1 can have a connecting bridge 40 positioned, based on its profile, on axis B either:
Tilted at angle α with regard to longitudinal axis A and offset in direction Dₛ₁ along axis YY′;
Titled at angle α with regard to longitudinal axis A and offset in direction Dₛ₂ along axis ZZ′;
Titled at angle α with regard to longitudinal axis A and offset in the combination of directions Dₛ₁ and/or Dₛ₂.

According to the invention, distances Dₛ₁ and Dₛ₂ are always above 0 and lower than or equal to 20 millimeters for Dₛ₁ and lower than or equal to 10 millimeters for Dₛ₂.

This combination of axial offsets of longitudinal axis B of the second part 30 helps prevent the offset of the outer and upper parts between the first bone P2 and the second bone P1 when implant 1 is inserted in their medullary canals.

According to the embodiment, longitudinal axis A of the first part 20 cuts longitudinal axis B of the second part 30 at a point of intersection I₃ located between the distal end 20a and the proximal end 20b of the said first part 20.

This point of intersection I₃ is located at a distance L₃ from the proximal end 20b and at a distance L₃ from the distal end 20a of the first part 20.

The point of intersection I₃ is spaced from the distal end 20a of the first part 20 in the proximal direction so as to be located in an area corresponding to the proximal half of the first part 20. Such embodiments make it possible to optimally offset the distal phalange P2 in the caudal direction from the proximal phalange P1 after insertion.

Also, the orthonormal axes X, Y Z of the connecting bridge 40 intersect at a point of intersection I₂ which is located at a distance L₂ from the proximal face 30a of the second part 30. Another part of the vertical axis YY′ of the connecting bridge 40 cuts the longitudinal axis B of the second part 30 at a point of intersection I₁, located at a distance Dₛ₁ from the point of intersection I₂. The longitudinal axis ZZ′ of the connecting bridge 40 also cuts the longitudinal axis B of the second part 30 at a point of intersection I₄ located at a distance Dₛ₂ from the point of intersection I₂.

In the variations shown here, the longitudinal axis XX′ of the connecting bridge 40 of the implant 1 can coincide with the longitudinal axis A of the first part 20 or be titled with regard to the longitudinal axis A of the first part 20.

Due to its specific structure, the implant 1 according to the invention, once implanted, offsets internally the distal phalange with regard to the proximal phalange. In particular, due to the implant according to the invention, the distal phalange, once fused with the proximal phalange, has a longitudinal axis that is slightly offset downward, in the caudal direction.

As such, any distal phalange is offset in the caudal direction with regard to an implant from prior art for which axes A and B are not separated by the distance Dₛ₁ and/or Dₛ₂ as defined above.

After the insertion of the implant according to the invention, the outer parts of the proximal phalange and the distal phalange will be perfectly aligned, and the surgeon does not need to cut the upper part of the bone of the distal phalange to realign the upper parts of the phalanges.

Indeed, because of the implant according to the invention, the upper part of the bone of the distal phalange does not exceed the upper line of the proximal phalange and this bone does not create a step between the proximal phalange and the distal phalange.

Also, because of the implant according to the invention, it is possible to fuse two phalanges P1 and P2 into one and block the joint between these two phalanges and treat pathologies such as claw toes, metatarsalgia or even Hallux Valgus.

1. Medical implant (1) intended to allow bone fusion between a first bone (P1) and a second bone (P2), the said implant comprising a first, elongated part (20), extending along the longitudinal axis A, intended to be inserted into the first bone (P1) and comprising first attachment means (24) and a second, also elongated part (30) on another longitudinal axis B, intended to be inserted into the second bone (P2) and comprising second attachment means (34), said first and second parts (20, 30) being connected by a connecting bridge (40), characterized in that the said connecting bridge (40) is centered around orthonormal axes X, Y, Z of the said implant (1), creating at least one axial offset of the second part (30) with regard to the first part (20) so that the longitudinal axis B of the second part (30) is positioned in at least one direction, titled at an angle (α) with regard to the longitudinal axis A of the first part (20), as well as offset by a length of at least one of the axes XX′, YY′, and ZZ′ of the connecting bridge (40) to prevent the creation of an offset of the upper exterior parts between the first bone (P2) and the second bone (P1).

2. Medical implant (1) according to claim 1, characterized in that the longitudinal axis B of the said second part (30) is offset by a distance (Dₛ₁) equal to the length of axis YY′.

3. Medical implant (1) according to claim 1, characterized in that the longitudinal axis B of the said second part (30) is offset by a distance (Dₛ₂) equal to the length of axis ZZ′.

4. Medical implant (1) according to claim 1, characterized in that the first part (20) is in an elongated shape and comprises at least three brackets (21, 22, 23) each respectively extending along the longitudinal axis A and including a distal end (20A) and a proximal end (20b).

5. Medical implant (1) according to claim 4, characterized in that the brackets (21, 22, 23) are regularly arranged around the longitudinal axis A, providing a space to drill a bore hole or canula (25) between the said brackets and in the body of the first part (20), supported by longitudinal axis A and crossing the connecting bridge (40) allowing the guide pin to fit through.

6. Medical implant (1) according to claim 4, characterized in that each bracket (21, 22, 23) comprises attachment means on its outer edges, made of hooked notches (24), creating a toothed external profile all along the first part (20). The said hooked notches (24) each have an edge (24a) pointing toward the connecting bridge (40).

7. Medical implant (1) according to claim 1, characterized in that the second part (30) is extended and comprises at least three brackets (31, 32, 33) each respectively extending along longitudinal axis B and comprising a distal end (30a) and a proximal end (30b).

8. Medical implant (1) according to claim 1, characterized in that the longitudinal axis A of the first part (20) cuts the longitudinal axis B of the second part (30) in a point of intersection I₃ located between the distal end (20a) and the proximal end (20b) of the said first part (20).

9. Medical implant (1) according to claim 4, characterized in that the point of intersection I₃ is located at a distance L₃ from the proximal end (20b) and a distance L₃ from the distal end (20a) of the first part (20).

10. Medical implant (1) according to claim 1, characterized in that the orthonormal axes X, Y, Z of the connecting bridge (40) intersect at a point of intersection I₂.

11. Medical implant (1) according to claim 5, characterized in that the point of intersection I₂ is located a distance L₂ from the proximal face (30a) of the second part (30).

12. Medical implant (1) according to claim 1, characterized in that the vertical axis D of the connecting bridge (40) intersects the longitudinal axis B of the second part (30) at a point of intersection I₁ is located at a distance Dₛ₁ from the point of intersection I₂.

13. Medical implant (1) according to claim 1, characterized in that the longitudinal axis XX′ of the connecting bridge (40) can coincide with the longitudinal axis A of the first part (20).

14. Medical implant (1) according to claim 1, characterized in that the longitudinal axis XX′ of the connecting bridge (40) is titled with regard to the longitudinal axis A of the first part (20).

15. Medical implant (1) according to claim 1, characterized in that the connecting bridge (40) is surrounded by two opposite faces (40, 41) that coincide respectively with the distal end (20a) of the first part (20) and the proximal end (30b) of the second part (30).

16. Medical implant (1) according to claim 2, characterized in that the distance (Dₛ₁) is less than or equal to 20 millimeters.

17. Medical implant (1) according to claim 3, characterized in that the distance (Dₛ₁) is less than or equal to 10 millimeters.
